(19) United States
(12) Patent Application Publication
(10) Pub. No.: US 2025/0235563 A1
(43) Pub. Date: Jul. 24, 2025

(54) NONTOXIC STAIN TO VISUALIZE BACTERIAL BIOFILM DURING IRRIGATION AND DEBRIDEMENT OF SURGICAL SITE INFECTIONS

(71) Applicant: Daniel Robert Schlatterer, Dunwoody, GA (US)

(72) Inventor: Daniel Robert Schlatterer, Dunwoody, GA (US)

(21) Appl. No.: 18/418,504

(22) Filed: Jan. 22, 2024

(51) Int. Cl. A61K 49/00

(52) U.S. Cl. CPC A61K 49/006; A61K 49/0032

(57) ABSTRACT

A nontoxic biofilm visualization stain can be used in a mammalian infected surgical site washout or irrigation and debridement to enhance the thoroughness and success rate of the surgical site infection washout or irrigation and debridement. The biofilm staining composition is a prokaryotic, non-eukaryotic stain to improve visualization of bacteria laden biofilm. Unbound biofilm staining composition can be removed from the surgical site at the end of the irrigation and debridement with saline irrigation, but it is not clinically mandatory.

### TECHNICAL FIELD

The present invention relates to an improved, faster, and more effective method of identifying bacteria laden biofilm in surgical site infections (SSI). The goal is to improve an SSI washout or an SSI irrigation and debridement (I&D) by improving visualization of where bacteria are located within an SSI. Improved bacteria visualization facilitates thoroughness of the I&D and in turn leads to a more effective SSI treatment with less operative time.

### BACKGROUND OF THE INVENTION

Orthopedic surgeons use a variety of implants to reconstruct or replace worn out hip, knee, shoulder, elbow, wrist and ankle joints. Orthopedic surgeons also use plates, screws, cages, and rods for fracture reduction and fixation, and spinal procedures. Unfortunately, a significant percentage of these procedures that use foreign bodies (implants) get a surgical site infection or SSI, and despite an I&D of the SSI, infections often recur. Recurrences are thought to be due to residual bacteria within biofilm at the surgical site.

Biofilm is a community of microbes producing and maintaining a complex network of polysaccharides and other carbon polymers. This impenetrable lattice provides protection to the bacteria community throughout an infected surgical site, in particular protection to bacteria within biofilm adhering to the surface of orthopedic implants. In addition to the physical barrier that biofilm affords bacteria, the microbes deep within biofilm are resistant to a range of antimicrobial agents including clinically proven and frequently used antibiotics. The combination of physical protection by biofilm and antibiotic resistance of microbes within biofilm emphasizes the importance of thorough biofilm removal versus antibiotic administration for SSI treatment. In other words, treating an SSI is more effective if all bacteria are removed from an infection site as opposed to treating with intravenous (IV) antibiotics which are ineffective on bacteria within biofilm. One challenge with washing out an SSI is that bacterial biofilm is slimy and very adherent to implants. The adhesiveness of biofilm prevents implant decontamination with saline irrigation. Chewing gum stuck to a floor is a relatively comparable situation. Therefore, effective cleaning of an infected surgical site has two primary steps. First, removal of all pus at the site via irrigation. Second, scrubbing the implants to remove all bacteria laden biofilm.

Thorough biofilm removal first requires accurate biofilm visualization in order to see what needs to be cleaned and removed. The concept of biofilm formation was first reported by Gristina and Webb in the journal Science in 1985. [Gristina, A. G., Oga, M., Webb, L. X., & Hobgood, C. D. (1985). Adherent bacterial colonization in the pathogenesis of osteomyelitis. Science, 228(4702), 990-993. PMID: 40019331.

These authors coined the phrase “The race to the surface” implying the race to the implant surface between bacteria and host cells. They also postulated that an implant is either encapsulated with fibrous tissue by the host's body, or a bacterial biofilm forms over the implant surface. This summarizes the “Race to the surface” concept proposed many years ago. This publication in 1985 and others since their landmark paper all point to the role biofilm plays in SSIs and infection recurrence. The pathway for infection recurrence after an SSI I&D is often a matter of incomplete biofilm removal because the biofilm contains bacteria that reemerge after the I/D and cause an infection recurrence. Moreover, the residual bacteria laden biofilm is not due to a lack of effort by the I&D surgeon. Rather, it is a function of the I&D surgeon not seeing where the biofilm exists and needs to be cleaned away. Therefore, biofilm that is not seen, is not removed during an SSI I&D and since it remains at the surgical site, an infection recurs despite after an I&D. This sequence of infection recurrences after I&D's again emphasizes the importance of accurate biofilm visualization to enable complete biofilm removal during an SSI I&D. A very aggressive SSI treatment method would include implant removal and an I&D of the SSI site. Unfortunately implant removal often damages the underlying bone during the explantation process. Therefore, implant removal is not always feasible because orthopedic implants are required to maintain structural integrity of the joint or fractured bone.

Recently, a more conservative SSI treatment method which preserves bone stock at the SSI site has emerged in the adult reconstruction subspecialty of orthopedics. The acronym for this SSI treatment method is DAIR or debridement antibiotics and implant retention. This method has a high infection recurrence rate theoretically due to retention of biofilm covered implants. In other words, the infected implants are retained and cleaned but not cleaned completely of biofilm. The solution to SSIs is twofold. First, improved infection prevention. Second is improved infection treatment methods. A nontoxic biofilm stain can be a useful adjunct during SSI I&Ds. The stain will enhance bacteria laden biofilm visualization, which will improve I&D's and improve SSI treatment.

Currently, no biofilm stain is approved for in vivo use due to their cytotoxicities. Ruthenium red is one example among other biofilm stains that are used in histology but are not approved for in vivo use.

### Definitions

As used herein and in the claims:

A biofilm is the extracellular polymeric substance (EPS) produced by bacteria at an orthopedic surgical site or other surgical site. A biofilm comprises any syntrophic consortium of microorganisms in which cells stick to each other and often also to an implant or body surface (bone, synovium, fascia or other human tissue). Bacterial cells become embedded within the biofilm which is a slimy/sticky extracellular matrix composed of extracellular polymeric substances (EPS). The cells within the biofilm produce the EPS components, which are typically a polymeric conglomeration of extracellular polysaccharides, proteins, lipids and genetic material. Biofilm has a protective and adaptive role in the life cycle of bacteria. Therefore, it is expected that over time, biofilm composition will evolve and change in response to environmental pressures. Therefore, the description of a nontoxic biofilm stain contained herein will differ in years to come. However, the purpose of a nontoxic biofilm stain will remain unchanged. It is expected that the biofilm stain described herein will change in the years to come to meet the intention of biofilm visualization.

A biofilm stain is specific to biofilm produced by prokaryotic cells, and will not stain or alter eukaryotic (human) cells. The cell species specificity of the biofilm stain described herein improves visualization of only bacteria laden biofilm. The stain will bind to molecules within biofilm such as glucose and glycogen which are unique to biofilms and are not found on any other human body cells or tissue surfaces. Glucose and glycogen are found within human cells but not on the surface of any human tissues. This is a unique opportunity to stain for glucose/glycogen and visualize biofilm and not any other human cells, structures or organs. Moreover, stains for glucose and glycogen are inert and nontoxic. Therefore, this stain is sensitive and specific for biofilm while being non cytoxic to human cells.

Fluorescein 5-isothiocyanate is the 5-isomer of fluorescein isothiocyanate. Acts as a fluorescent probe capable of being conjugated to tissue and proteins; used as a label in fluorescent antibody staining procedures as well as protein- and amino acid-binding techniques.

Food coloring dye or stain may include: food coloring dye yellow #5 (Tartrazine) or yellow #6 (Sunset Yellow).

3,3′,5,5′-Tetramethylbenzidine or TMB is a chromogenic substrate used in staining procedures in immunohistochemistry as well as being a visualizing reagent used in enzyme-linked immunosorbent assays (ELISA).

### SUMMARY OF THE INVENTION

A biofilm staining composition has fluorescein 5-isothiocyanate (FITC); nontoxic coloring dye or stain; and wherein the FITC and the nontoxic coloring dye or stain are added to a volume of sterile water to form the biofilm staining composition that will bind to a biofilm and be visualized due to the nontoxic coloring dye or stain in the composition, and wherein the composition reacts with amine groups to form conjugates with proteins for visual detection of the biofilm by seeing with the naked eye where the composition is located in the surgical site infection.

The biofilm staining composition is a prokaryotic, non-eukaryotic stain to improve visualization of bacterial biofilm. The biofilm staining composition binds to the biofilm, the biofilm having one or more bacteria stainable by the biofilm staining composition, the bacteria being one or more of; Pseudomonas aeruginosa, Staphylococcus epidermidis, Streptococcus veridans, Klebsiella pneumoniae, Proteus mirabilis, Escherichia coli (E. coli), Staphylocossus aureus, or Enterococcus faelcalis.

The biofilm staining composition is configured to target other biofilm forming agents. The other biofilm forming agents include viruses, fungi, bacteria or subspecies thereof, or other infectious agents. The nontoxic coloring dye or stain is in the form of a biofilm color or a biofilm stain that changes color upon binding to the biofilm. The nontoxic coloring dye or stain is reactive to the FITC when bound to the biofilm initiating the change in color.

A method of improving biofilm visualization during treatment of a surgical site infection has the steps of: exposing a surgical site having a surgical site infection; applying 5-10 ml of the biofilm staining composition to the surgical site infection, thereby staining the biofilm for visual detection; debriding surgical site infection until all visualized biofilm is removed; irrigating the surgical site, washing away the biofilm staining solution; and closing the surgical site. The step of irrigating further comprises removing excess biofilm staining composition from the surgical site with saline or other aqueous solution. The step of applying further comprises an oxidation step that may be required dependent upon the biofilm reagent. The method further comprises the step of washing away the biofilm staining composition.

A second embodiment nontoxic biofilm staining composition has Amplex red, 3,3′,5,5′-Tetramethylbenzidine (TMB) or fluorescein 5-isothiocyanate (FITC); nontoxic coloring dye or stain; and wherein the Amplex red, 3,3′,5,5′-Tetramethylbenzidine (TMB) or fluorescein 5-isothiocyanate (FITC) and the nontoxic coloring dye or stain are added to a volume of sterile water to form the biofilm staining composition that will bind to a biofilm and be visualized due to the nontoxic food coloring dye or stain in the solution, and wherein the solution reacts with amine groups to form conjugates with biofilm proteins for visual detection of the biofilm by seeing with the naked eye where the solution with the nontoxic food coloring dye or stain is located within the SSI.

The biofilm staining solution binds to specific biofilm components and thus visually demonstrates biofilm location. The biofilm staining solution reacts with amine groups to form conjugates with proteins for visual detection of the biofilm. The FITC reaction is only a covalent binding process. There are no secondary by-products of the binding and staining process using FITC or any other staining materials ingredients or reagents. Alternatively, the glucose and glycogen within biofilm can be visualized by the photooxidation of Amplex red to resorufin. This oxidation process has 2 steps. First, Amplex red is applied by an aerosolized spray to the SSI site. Second, horseradish peroxidase is sprayed over the areas having Amplex red. These 2 steps result in a pinkish coloration of the biofilm due to the oxidation of Amplex red. Another reagent to detect glucose and glycogen on biofilm includes 3,3′5,5′-tetra methyl benzide (TMB). An evolution of the glucose and glycogen staining steps is anticipated as clinical experience dictates to achieve the best biofilm visualization with naked eyes. In other words, the three methods listed herein (FITC, Amplex red and TMB) are nontoxic biofilm staining examples and other methods with other reagents will likely emerge. Additional biofilm detection methods may employ latex particles, nanoparticles and or fluorescein. The three above staining methods have been included for illustrative purposes only. The methods presented herein may not be currently comprehensive nor are they expected to be comprehensive in the future. The final staining reagents will be worked out with and ultimately approved by the FDA. Inclusion of these three staining reagents is for illustrative purposes. In order to demonstrate how a nontoxic biofilm stain will be achieved.

The FITC with the nontoxic food coloring dye or stain binds to the biofilm, the biofilm having one or more bacteria stainable by the FITC with the nontoxic coloring dye, the bacteria being one or more of; Pseudomonas aeruginosa, Staphylococcus epidermidis, Streptococcus veridans, Klebsiella pneumoniae, Proteus mirabilis, Escherichia coli (E. coli), Staphylocossus aureus, or Enterococcus faelcalis.

The FITC has food coloring added to it so that once it binds to biofilm it can be readily visualized. Unlike Amplex red or TMB, FITC doesn't require oxidation to visualize its location. The biofilm staining composition described herein is configured to target other biofilm forming agents. The other biofilm forming agents include viruses, fungi, bacteria or subspecies thereof, or other infectious agents.

### DETAILED DESCRIPTION OF THE INVENTION

The present invention relies on the biochemical structural properties of biofilm commonly produced by eight different bacteria; Pseudomonas aeruginosa, Staphylococcus epidermidis, Streptococcus veridans, Klebsiella pneumoniae, Proteus mirabilis, Escherichia coli (E. coli), Staphylocossus aureus, and Enterococcus faelcalis among others. For example, components the biofilm matrix contain include glucose, glycogen, extracellular proteins, cell surface adhesins and protein subunits of cell appendages such as flagella and pili. Specific examples of these proteins include RbmA, Bap1,1ECb and RbmC. These proteins are important for biofilm formation on abiotic surfaces. Additionally, this invention could also target quorum sensing proteins such as AHLs (N-acyl homoserine lactone) secreted by bacteria into their biofilm. All of these stainable entities are unique to prokaryotes. Thus, this invention will not target any eukaryotic or human tissue components. Moreover, the stain is not cytotoxic to bacterial cells, and no cellular debris or toxic byproducts result from the staining steps. The stain binds to biofilm protein components covalently which permits sustained visualization of biofilm loci during an SSI I&D regardless of blood or irrigation entering the SSI site during the I/D procedure. The stain will be composed of Amplex red, TMB, FITC, or other reagents yet to be identified. FITC reacts with amine groups while Amplex red, and TMB reacts with glucose or glycogen to form conjugates for naked eye detection. In this iteration, the FITC will be mixed with a nontoxic food coloring agent. The colored FITC will be easily visualized once it binds to the biofilm. Saline irrigation will not remove the covalently bound FITC or the food coloring dye in the FITC. A fluorescent detecting lens is not required for visualization of the FITC bound to biofilm because food coloring dye is added to the FITC. The FITC will irreversibly bind to the biofilm and will be easily visualized due to the inert food coloring dye added to it.

Implant related surgical site infections especially in orthopedics have biofilms within the infected site.

This invention stains biofilm which improves bacterial laden biofilm visualization and removal during an SSI I&D because the biofilm can be seen. Bacteria whose biofilm is stainable by this invention: Pseudomonas aeruginosa, Staphylococcus epidermidis, Streptococcus veridans, Klebsiella pneumoniae, Proteus mirabilis, Escherichia coli (E. coli), Staphylocossus aureus, Enterococcus faelcalis among others.

A method of improving biofilm visualization during an SSI I&D comprising the steps of: a) Standard surgical exposure of the SSI. b) Spray-bottle application (5-10 ml) of the biofilm stain to the infected SSI followed by the possible application of an oxidizing second reagent to Amplex red or TMB c) Debridement of the infected surgical site until all visualized biofilm is removed. The exact method of stained biofilm removal is at the discretion of the surgeon. Previously, a similar debridement technique using the application of methylene blue to enhance debridements was reported and is applicable herein as the debridement method. [Shaw, J. D., Miller, S., Plourde, A., Shaw, D. L., Wustrack, R., & Hansen, E. N. (2017). Methylene Blue-Guided Debridement as an Intraoperative Adjunct for the Surgical Treatment of Periprosthetic Joint Infection. The Journal of Arthroplasty, 32(12), 3718-37231 d) the debrided infected surgical site will be closed per surgeon protocol. e) once the biofilm-stained debrided SSI wound is closed, nothing further is required specific to the use of the biofilm stain. It is recommended that excess stain from the site is irrigated away with saline before closure, but it is not clinically required. The stain is nontoxic to all cells and may remain within the SSI site at the completion of its use to visualize biofilm. Biofilm stain remaining within a SSI after its debridement is not intended to provide any treatment effect as do other currently available pre-closure surgical washes.

Variations in the present invention are possible in light of the description of it provided herein. The chemical composition of the biofilm stain may change as new biofilm information emerges and as biofilm evolves due to environmental pressures. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims. The surgical access window described herein encompasses the dimensions presented and any and all variations applicable to the methods and surgical technique described directly or indirectly intended with this device.

What is claimed is:

1. A biofilm staining composition comprises:
fluorescein 5-isothiocyanate (FITC);
nontoxic coloring dye or stain; and
wherein the FITC and the nontoxic coloring dye or stain are added to a volume of sterile water to form the biofilm staining composition that will bind to a biofilm and be visualized due to the nontoxic coloring dye or stain in the composition, and wherein the composition reacts with amine groups to form conjugates with proteins for visual detection of the biofilm by seeing with the naked eye where the composition is located in the surgical site infection.

2. The biofilm staining composition of claim 1, wherein the biofilm staining composition is a prokaryotic, non-eukaryotic stain to improve visualization of bacterial biofilm.

3. The biofilm staining composition of claim 1, wherein the biofilm staining composition binds to the biofilm, the biofilm having one or more bacteria stainable by the biofilm staining composition, the bacteria being one or more of; Pseudomonas aeruginosa, Staphylococcus epidermidis, Streptococcus veridans, Klebsiella pneumoniae, Proteus mirabilis, Escherichia coli (E. coli), Staphylocossus aureus, or Enterococcus faelcalis among others.

4. The biofilm staining composition of claim 1, wherein the biofilm staining composition is configured to target other biofilm forming agents, microbes, or organisms.

5. The biofilm staining composition of claim 4, wherein the other biofilm forming agents include viruses, fungi, bacteria or subspecies thereof, or other infectious agents.

6. The biofilm staining composition of claim 1, wherein the nontoxic coloring dye or stain is in the form of a biofilm color or a biofilm stain that may change color upon binding to the biofilm or oxidation or by another secondary reagent mechanism.

7. The biofilm staining composition of claim 6, wherein the biofilm staining composition when bound to the biofilm initiates the change in color.

8. A method of improving biofilm visualization during a treatment of a surgical site infection comprising the steps of:
exposing a surgical site having a surgical site infection;
applying 5-10 ml of a biofilm staining composition to the surgical site infection, thereby staining the biofilm on implants and the surgical site for biofilm visual detection;
debriding surgical site infection until all visualized biofilm is removed;
irrigating the surgical site, thereby removing the debrided biofilm; and
closing the surgical site.

9. The method of claim 8, wherein the step of irrigating further comprises removing excess biofilm staining composition from the surgical site with saline or other aqueous solution.

10. The method of claim 8, wherein the step of applying further comprises an oxidation step or other secondary reagent application that may be required dependent upon the biofilm reagent.

11. The method of claim 8, further comprises the step of washing away the biofilm staining composition per surgeon preference, but not clinically mandatory.

12. A biofilm staining composition comprises:
Amplex red, 3,3′,5,5′-Tetramethylbenzidine (TMB) or fluorescein 5-isothiocyanate (FITC);
nontoxic coloring dye or stain; and
wherein the Amplex red, 3,3′,5,5′-Tetramethylbenzidine (TMB) or fluorescein 5-isothiocyanate (FITC) and the nontoxic coloring dye or stain are added to a volume of sterile water to form the biofilm staining composition that will bind to a biofilm and be visualized due to the nontoxic food coloring dye or stain in the solution, and wherein the solution reacts with amine groups to form conjugates with proteins for visual detection of the biofilm by seeing with the naked eye where the solution with the nontoxic food coloring dye or stain is located within the SSI.

13. The biofilm staining composition of claim 12, wherein Amplex red or TMB require an oxidation step with horseradish peroxidase (HRPO) to provide visualization, the oxidation step will turn Amplex red to a pinkish color.

14. The biofilm staining composition of claim 12, wherein the biofilm staining composition is a prokaryotic, non-eukaryotic stain to improve visualization of bacterial biofilm.

15. The biofilm staining composition of claim 12, wherein the biofilm staining composition binds to the biofilm, the biofilm having one or more biofilm producing bacteria stainable by the biofilm staining composition, the bacteria being one or more of; Pseudomonas aeruginosa, Staphylococcus epidermidis, Streptococcus veridans, Klebsiella pneumoniae, Proteus mirabilis, Escherichia coli (E. coli), Staphylocossus aureus, or Enterococcus faelcalis among others.

16. The biofilm staining composition of claim 12, wherein the biofilm staining composition is configured to target other biofilm forming agents, microbes or organisms.

17. The biofilm staining composition of claim 16, wherein the other biofilm forming agents include viruses, fungi, bacteria or subspecies thereof, or other infectious agents.

18. The biofilm staining composition of claim 12, wherein the nontoxic food coloring dye or stain is in the form of a biofilm color or a biofilm stain that changes color upon binding to the biofilm or after an oxidation step.

19. The biofilm staining composition of claim 18, wherein the nontoxic coloring dye or stain is mixed with the FITC permitting naked eye visualization of biofilm.

20. The biofilm staining composition of claim 18, will visualize the biofilm or extracellular polymeric substance (EPS) formed by agents, microbes or organisms other than bacteria, the biofilm and EPS formed by agents, microbes or organisms other than bacteria may one day be referred to as something other than biofilm or EPS, nonetheless, these other agents, microbes, and organisms other than bacteria are expected to secrete cellular products which may not technically be biofilm but will remain as extracellular polymeric substances (EPS) detectable and potentially visualized by specific nontoxic EPS stains.
